(19) United States
(12) Patent Application Publication
(10) Pub. No.: US 2026/0240760 A1
(43) Pub. Date: Aug. 20, 2026

(54) Injectable Compositions of Vitamers of Vitamin B12 and Uses Thereof

(71) Applicant: Nevakar Injectables Inc., Bridgewater, NJ (US)

(72) Inventors: Harshil H. Jain, Monmouth Junction, NJ (US); Kumaresh Soppimath, Skillman, NJ (US); Tushar Hingorani, Bridgewater, NJ (US)

(73) Assignee: Nevakar Injectables Inc., Bridgewater, NJ (US)

(21) Appl. No.: 19/160,650

(22) PCT Filed: Mar. 1, 2024
(86) PCT No.: PCT/US2024/018134

§ 371 Date: Aug. 28, 2025

Related U.S. Application Data
(60) Provisional application No. 63/487,665, filed on Mar. 1, 2023.

(51) Int. Cl. A61K 9/00; A61K 9/107; A61K 9/1277; A61K 31/714; A61K 47/24; A61K 47/59

(52) U.S. Cl. CPC A61K 9/0019; A61K 9/1075; A61K 9/1277; A61K 31/714; A61K 47/24; A61K 47/593

(57) ABSTRACT

The present disclosure is directed to novel long acting and sustained release compositions comprising vitamers of vitamin B12 and methods for treating vitamin B12 deficiency using said compositions.

### FIELD AND BACKGROUND OF THE INVENTION

The present disclosure relates to long-acting and sustained release injectable compositions comprising vitamers of vitamin B12. The present disclosure also relates to methods of treating vitamin B12 deficiency in a subject in need thereof.

Cyanocobalamin, hydroxocobalamin, adenosylcobalamin, and methylcobalamin are vitamers of vitamin B12 that can be used to treat vitamin B12 deficiency, with cyanocobalamin being the most convenient form to produce industrially.

Vitamin B12 deficiency can occur for a variety of reasons, including Addisonian (pernicious) anemia, gastrointestinal pathology, gastrointestinal dysfunction, or gastrointestinal surgery, fish tapeworm infestation, malignancy of pancreas or bowel, folic acid deficiency, a vegetarian diet, a vegan diet, or a combination of any of these conditions. In order to treat this deficiency, injectable or intranasal compositions comprising vitamin B12 have been developed. Intranasal compositions lead to highly variable and unpredictable vitamin B12 uptake, and are therefore only used in select circumstances. Injectable compositions are the current standard of care but suffer from low vitamin B12 uptake in subjects, with as much as 98% of vitamin B12 excreted in urine within 48 hours of injection. This necessitates monthly administration on a permanent basis. This frequency and method of administration can be undesirable to patients, leading to noncompliance and poor patient outcomes.

There is a need for vitamin B12 compositions that are capable of slowly releasing vitamin B12 over a period of time allowing administration only once every 3-9 months or multiple weeks. In addition, there is a need for vitamin B12 salts that dissolve significantly more effectively and release vitamin B12 over an extended period of time.

### BRIEF SUMMARY OF THE INVENTION

### Pharmaceutical Compositions

The present disclosure provides long-acting and sustained release injectable compositions comprising vitamers of vitamin B12.

In some aspects, the composition is a pharmaceutical long-acting injectable depot composition comprising a vitamer of vitamin B12 or a pharmaceutically acceptable derivative thereof, wherein the composition is dispersed in a biodegradable carrier.

In some aspects, the vitamer is selected from the group consisting of cyanocobalamin, hydroxocobalamin, adenosylcobalamin, and methylcobalamin.

In some aspects, the vitamer is cyanocobalamin.

In some aspects, the vitamer is a pharmaceutically acceptable salt.

In some aspects, the pharmaceutically acceptable salt is an organic salt.

In some aspects, the organic salt is a pamoate salt, a palmitate salt, an oleate salt, an octanoate salt, a decanoate salt, a hexanoate salt, or a stearate salt.

In some aspects, the vitamer is cyanocobalamin pamoate.

In some aspects, the biodegradable carrier is selected from the group consisting of a lipid, an oil, a polymer, and combinations and mixtures thereof.

In some aspects, the polymer is selected from the group consisting of polylactides, polyglycolides, poly(lactide-co-glycolide) s, polycaprolactones, polydioxanones, polycarbonates, polyhydroxybutyrates, poly(3-hydroxybutyrate-co-3-hydroxyvalerate), tri (ethylene glycol) poly(orthoester), polyalkylene oxalates, polyanhydrides, polyamides, polyesteramides, polyurethanes, polyacetals, polyorthocarbonates, polyphosphazenes, polyhydroxyvalerates, polyalkylene succinates, polyorthoesters, and copolymers, block copolymers, branched copolymers, terpolymers, sucrose acetate isobutyrate (SAIB), and combinations and mixtures thereof.

In some aspects, the polymer is a polylactide, a poly(lactide-co-glycolide), or a combination thereof.

In some aspects, the poly(lactide-co-glycolide) has a lactide to glycolide ratio of from about 5:95 to about 95:5.

In some aspects, the poly(lactide-co-glycolide) has a lactide to glycolide ratio of about 50:50.

In some aspects, the polymer has an intrinsic viscosity of from about 0.1 dL/g to about 1 dL/g.

In some aspects, the polymer has an intrinsic viscosity of about 0.25 dL/g.

In some aspects, the polymer has a molecular weight of from about 5 kDa to about 100 kDa.

In some aspects, the polymer has a molecular weight of from about 15 kDa to about 35 kDa.

In some aspects, the polymer has a molecular weight of about 25 kDa.

In some aspects, the concentration of polymer is from about 10% to about 40%.

In some aspects, the concentration of polymer is about 30%.

In some aspects, the lipid is selected from the group consisting of phosphatidylcholines, phoshatidylethanolamines, phosphatidic acids, phosphatidylinositols, phosphatidylserines, phosphatidylglycerols, diphosphatidylglycerols, sterols, and combinations and mixtures thereof.

In some aspects, the oil is selected from the group consisting of medium chain triglycerides of caprylic (C8) and capric (C10) acids, any pure fatty acid ester such as ethyl, propyl isopropyl, and butyl; esters of fatty acids such as caproic, caprylic, capric, lauric, palmitic, myristic, and stearic acids, isopropyl myristate, isopropyl palmitate, isopropyl caproate, isopropyl caprylate, ethyl stearate, butyl laurate, coconut oil, palm kernel oil, soy bean oil, castor oil, cotton seed oil, corn oil, and olive oil, and combinations and mixtures thereof.

In some aspects, the composition is stable for at least about 6 months at 25° C.

In some aspects, the composition is stable for at least about 9 months at 25° C.

In some aspects, the composition is stable for at least about 12 months at 25° C.

In some aspects, the composition is stable for at least about 18 months at 25° C.

In some aspects, the composition is lyophilized into a solid powder.

In some aspects, the amount of vitamer of vitamin B12 is from about 0.1 mg to about 5 mg.

In some aspects, the amount of vitamer of vitamin B12 is from about 0.5 mg to about 3 mg.

In some aspects, the amount of vitamer of vitamin B12 is from about 0.7 mg to about 1 mg.

In some aspects, the amount of vitamer of vitamin B12 is about 0.8 mg.

In some aspects, the amount of vitamer of vitamin B12 is about 2 mg.

In some aspects, the concentration of the vitamer of vitamin B12 is from about 1 mg/g to about 6 mg/g.

In some aspects, the concentration of the vitamer of vitamin B12 is about 4 mg/g.

In some aspects, the composition has a pH of from about 5 to about 8.

In some aspects, the pH is from about 6.5 to about 7.5.

In some aspects, the composition comprises one or more pharmaceutically acceptable excipients are selected from the group consisting of preservatives, buffers, bulking agents, tonicity agents, sugars, wetting agents, lubricants, emollients, antioxidants, co-solvents, surfactants, stabilizers, penetration enhancers, salts, osmotic agents, chelating agents, and resuspension components, or combinations thereof.

In some aspects, the solvent is a non-aqueous solvent.

In some aspects, the non-aqueous solvent is an organic solvent.

In some aspects, the organic solvent is a water-miscible solvent.

In some aspects, the water-miscible solvent is selected from the group consisting of dimethyl sulfoxide (DMSO), N-methyl-2-pyrrolidone (NMP), triacetin, propylene glycol, and combinations thereof.

In some aspects, the water-miscible solvent is DMSO or NMP.

In some aspects, the organic solvent is a water-immiscible solvent.

In some aspects, the water-immiscible solvent is selected from the group consisting of benzyl alcohol, benzyl benzoate, and combinations thereof.

In some aspects, the composition is formulated for parenteral (i.e., injectable) administration. In some aspects, the composition is formulated for subcutaneous administration. In some aspects, the composition is formulated for intramuscular administration.

In some aspects, the composition is a sustained release microparticle composition comprising i) a biocompatible polymer, ii) a vitamer of vitamin B12 or a pharmaceutically acceptable derivative thereof, and iii) one or more pharmaceutically acceptable excipients.

In some aspects, the vitamer of vitamin B12 is selected from the group consisting of cyanocobalamin, hydroxocobalamin, adenosylcobalamin, and methylcobalamin.

In some aspects, the vitamer of vitamin B12 is cyanocobalamin.

In some aspects, the biocompatible polymer is selected from the group consisting of polylactides, polyglycolides, poly(lactide-co-glycolide) s, polycaprolactones, polydioxanones, polycarbonates, polyhydroxybutyrates, polyalkylene oxalates, polyanhydrides, polyamides, polyesteramides, polyurethanes, polyacetals, polyorthocarbonates, polyphosphazenes, polyhydroxyvalerates, polyalkylene succinates, polyorthoesters, sucrose acetate isobutyrate (SAIB), and copolymers, block copolymers, branched copolymers, terpolymers, and combinations and mixtures thereof.

In some aspects, the composition is stable for at least about 6 months at 25° C.

In some aspects, the composition is stable for at least about 9 months at 25° C.

In some aspects, the composition is stable for at least about 12 months at 25° C.

In some aspects, the composition is stable for at least about 18 months at 25° C.

In some aspects, the composition is lyophilized into a solid powder.

In some aspects, the amount of vitamer of vitamin B12 is from about 0.1 mg to about 5 mg.

In some aspects, the amount of vitamer of vitamin B12 is from about 0.5 mg to about 3 mg.

In some aspects, the amount of vitamer of vitamin B12 is from about 0.7 mg to about 1 mg.

In some aspects, the amount of vitamer of vitamin B12 is about 0.8 mg.

In some aspects, the amount of vitamer of vitamin B12 is about 2 mg.

In some aspects, the composition has a pH of from about 5 to about 8.

In some aspects, the pH is from about 6.5 to about 7.5.

In some aspects, the one or more pharmaceutically acceptable excipients are selected from the group consisting of preservatives, buffers, bulking agents, tonicity agents, sugars, wetting agents, lubricants, emollients, antioxidants, co-solvents, surfactants, stabilizers, penetration enhancers, salts, osmotic agents, chelating agents, and resuspension components, or combinations thereof.

In some aspects, the composition comprises less than 1 wt % chlorinated solvents.

In some aspects, the microparticles have a mean diameter of from about 0.5 μm to about 1000 μm.

In some aspects, the microparticles have a mean diameter of from about 0.5 μm to about 100 μm.

In some aspects, the microparticles have a mean diameter of from about 100 μm to about 500 μm.

In some aspects, the microparticles have a mean diameter of from about 500 μm to about 1000 μm.

In some aspects, the biocompatible polymer has a molecular weight of from about 5 kDa to about 200 kDa.

In some aspects, the biocompatible polymer has a molecular weight of from about 5 kDa to about 100 kDa.

In some aspects, the biocompatible polymer has a molecular weight of from about 10 kDa to about 100 kDa.

In some aspects, the polydispersity index of the microparticles is from about 1.5 to about 2.5.

In some aspects, the ratio (g/g) of vitamer to polymer is from about 0.01 to about 0.1.

In some aspects, the composition is formulated for parenteral administration.

In some aspects, the composition is dispersed in an oil.

In some aspects, the oil is selected from the group consisting of medium chain triglycerides of caprylic (C8) and capric (C10) acids, any pure fatty acid ester such as ethyl, propyl isopropyl, and butyl; esters of fatty acids such as caproic, caprylic, capric, lauric, palmitic, myristic, and stearic acids, isopropyl myristate, isopropyl palmitate, isopropyl caproate, isopropyl caprylate, ethyl stearate, butyl laurate, coconut oil, palm kernel oil, soy bean oil, castor oil, cotton seed oil, corn oil, and olive oil, and combinations and mixtures thereof.

In some aspects, the composition is a long-acting liposomal composition comprising a lipid layer defining an internal aqueous compartment, a vitamer of vitamin B12 or a pharmaceutically acceptable derivative thereof, and at least one pharmaceutically acceptable excipient.

In some aspects, the vitamer is selected from the group consisting of cyanocobalamin, hydroxocobalamin, adenosylcobalamin, and methylcobalamin.

In some aspects, the vitamer is cyanocobalamin.

In some aspects, the lipid layer comprises a lipid selected from the group consisting of phosphatidylcholines, phoshatidylethanolamines, phosphatidic acids, phosphatidylinositols, phosphatidylserines, phosphatidylglycerols, diphosphatidylglycerols, sterols, and combinations and mixtures thereof.

In some aspects, the lipid layer comprises a surface stabilizing hydrophilic polymer-lipid conjugate.

In some aspects, the polymer-lipid conjugate comprises polyethylene glycol.

In some aspects, the liposome is a large unilamellar vesicle, a multilamellar vesicle, a small unilamellar vesicle, an interdigitating fusion liposome, or a mixture thereof.

In some aspects, the composition is stable for at least about 6 months at 25° C.

In some aspects, the composition is stable for at least about 9 months at 25° C.

In some aspects, the composition is stable for at least about 12 months at 25° C.

In some aspects, the composition is stable for at least about 18 months at 25° C.

In some aspects, the composition is lyophilized into a solid powder.

In some aspects, the composition is formulated for parenteral administration.

In some aspects, the composition is a long-acting microemulsion composition comprising a discontinuous internal phase comprising: an internal emulsifier, an aqueous solution, and a vitamer of vitamin B12 or a pharmaceutically acceptable derivative thereof; a continuous oil phase encompassing the internal phase; and an external emulsifier encompassing an oil phase.

In some aspects, the vitamer is selected from the group consisting of cyanocobalamin, hydroxocobalamin, adenosylcobalamin, and methylcobalamin.

In some aspects, the vitamer is cyanocobalamin.

In some aspects, the internal emulsifier is selected from the group consisting of propylene glycol monocaprylate or any other surfactant with an HLB value of from about 3 to about 7 and/or propylene glycol ester of any fatty acid such as propylene glycol monocaproate, propylene glycol monocaprylate, propylene glycol monocaprate, propylene glycol monolaurate, propylene glycol monostearate, propylene glycol monopalmitate, polyethylene glycol lauryl ether, polyethylene glycol oleyl ether, polyethylene glycol hexadecyl ether, sorbitan monopalmitate, sorbitan monostearate, sorbitan monooleate, sorbitan monolaurate, transcutol P, gelucire 50/13, gelucire 44/14, gelucire 43/01, any PEG mono-, di- and/or tri-esters of any fatty acid, lecithin, egg lecithin, phosphatidylcholine, phosphatidylethanolamine, phosphatidylinositol, tocopherol or any other phospholipid, and combinations thereof.

In some aspects, the oil phase is selected from the group consisting of medium chain triglycerides of caprylic (C8) and capric (C10) acids, any pure fatty acid ester such as ethyl, propyl isopropyl, and butyl; esters of fatty acids such as caproic, caprylic, capric, lauric, palmitic, myristic, and stearic acids, isopropyl myristate, isopropyl palmitate, isopropyl caproate, isopropyl caprylate, ethyl stearate, butyl laurate, coconut oil, palm kernel oil, soy bean oil, castor oil, cotton seed oil, corn oil, and olive oil, and combinations thereof.

In some aspects, the external emulsifier is selected from the group consisting of caprylo-caproyl polyoxyl-8 glycerides, macrogolglycerol ricinoleate, any other hydrophilic surfactant with a Hydrophile Lipophile Balance (HLB) value of from about 10 to about 16, polyethylene glycol mono- and/or di-esters of any fatty acid or fatty acid mixture, propylene glycol or any other alcohol including but not limited to glycerol, polyethylene glycol, ethanol, propanol, and isopropanol, and combinations thereof.

In some aspects, the composition is stable for at least about 6 months at 25° C.

In some aspects, the composition is stable for at least about 9 months at 25° C.

In some aspects, the composition is stable for at least about 12 months at 25° C.

In some aspects, the composition is stable for at least about 18 months at 25° C.

In some aspects, the composition is formulated for parenteral administration.

### Methods of Treatment

The present disclosure also provides methods of treating vitamin B12 deficiency in a subject, the method comprising administering to the subject a therapeutically effective amount of one or more of the long acting and sustained released injectable compositions described herein.

In some aspects, the subject's vitamin B12 levels remain at therapeutically effective levels over a period of about two to about six months.

In some aspects, the administration is parenteral.

In some aspects, the administration is intramuscular.

In some aspects, the administration is subcutaneous.

In some aspects, the composition is administered once every 0.5 to 6 months.

In some aspects, the composition is administered once every 3 months.

In some aspects, the composition is administered once every 6 months.

In some aspects, less than 50% of the cyanocobalamin administered to the subject is excreted in urine.

In some aspects, the composition provides a therapeutic dose of cyanocobalamin for at least 12 weeks.

In some aspects, the composition provides a therapeutic dose of cyanocobalamin for at least 16 weeks.

In some aspects, the composition provides a therapeutic dose of cyanocobalamin for at least 20 weeks.

In some aspects, the composition provides a therapeutic dose of cyanocobalamin for at least 24 weeks.

In some aspects, the vitamin B12 deficiency is a result of Addisonian (pernicious) anemia, gastrointestinal pathology, gastrointestinal dysfunction, or gastrointestinal surgery, fish tapeworm infestation, malignancy of pancreas or bowel, folic acid deficiency, vegetarian diet, vegan diet, or a combination of any of these conditions.

### BRIEF DESCRIPTION OF THE DRAWINGS

FIG. 1 is a line graph showing the release of cyanocobalamin over time for compositions comprising different polymers at a concentration of 25%.

FIG. 2 is a line graph showing the release of cyanocobalamin over time for compositions comprising Viatel 5002-A polymer at a concentration of 30%.

FIG. 3 is a line graph showing the release of cyanocobalamin over time for compositions comprising Viatel 5003-A polymer at a concentration of 30%.

FIG. 4 is a line graph showing the release of cyanocobalamin over time for compositions comprising Viatel 6503-A polymer at a concentration of 30%.

FIG. 5 is a line graph showing the release of cyanocobalamin over time for compositions comprising polylactic acid polymer at a concentration of 30%.

FIG. 6 is a line graph showing the release of cyanocobalamin over time for compositions comprising Viatel 5003-A polymer at varying concentrations.

### DETAILED DESCRIPTION OF THE INVENTION

### Definitions

Unless defined otherwise, all technical and scientific terms used herein have the same meaning as commonly understood by one of ordinary skill in the art to which this disclosure belongs. In case of conflict, the present application including the definitions will control. Unless otherwise required by context, singular terms shall include pluralities and plural terms shall include the singular. All publications, patents and other references mentioned herein are incorporated by reference in their entireties for all purposes as if each individual publication or patent application were specifically and individually indicated to be incorporated by reference.

Although methods and materials similar or equivalent to those described herein can be used in practice or testing of the present disclosure, suitable methods and materials are described below. The materials, methods and examples are illustrative only and are not intended to be limiting. Other features and advantages of the disclosure will be apparent from the detailed description and from the claims.

In order to further define this disclosure, the following terms and definitions are provided.

The singular forms “a,” “an” and “the” include plural referents unless the context clearly dictates otherwise. The terms “a” (or “an”), as well as the terms “one or more,” and “at least one” can be used interchangeably herein. In certain aspects, the term “a” or “an” means “single.” In other aspects, the term “a” or “an” includes “two or more” or “multiple.”

The term “about” is used herein to mean approximately, roughly, around, or in the regions of. When the term “about” is used in conjunction with a numerical range, it modifies that range by extending the boundaries above and below the numerical values set forth. In general, the term “about” is used herein to modify a numerical value above and below the stated value by a variance of 10 percent, up or down (higher or lower).

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The term “pharmaceutically acceptable” as used herein refers to those compounds, materials, compositions, formulations, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “excipient” refers to any substance, not itself a therapeutic agent, which may be used in a composition for delivery of an active therapeutic agent to a subject or combined with an active therapeutic agent (e.g., to create a pharmaceutical composition) to improve its handling or storage properties or to permit or facilitate formation of a dose unit of the composition. The excipient can be an inert substance, an inactive substance, and/or a not medicinally active substance.

The terms “effective amount” or “pharmaceutically effective amount” or “therapeutically effective amount” as used herein refer to the amount or quantity of a drug or pharmaceutically active substance which is sufficient to elicit the required or desired therapeutic response, or in other words, the amount which is sufficient to elicit an appreciable biological response when administered to a patient.

The term “essentially free” means that only traces amounts of other substances, or a reference substance (such trace amounts not having a substantial effect in the application), can be detected.

“Administration”, or “to administer” means the step of giving (i.e. providing) a pharmaceutical composition to a subject.

The term “pharmaceutically acceptable salts” is art-recognized, and includes relatively non-toxic, inorganic and organic acid addition salts of compounds and relatively non-toxic, inorganic and organic base addition salts of compounds.

The term “unit dosage form” or “unit dose composition” as used herein refers to a quantity of a compound, said quantity being such that one or more predetermined units may be provided as a single therapeutic administration.

As used herein, “treat”, “treatment”, and “treating” refer to the reduction or amelioration of the progression, severity, and/or duration of a given disease resulting from the administration of one or more therapies (including, but not limited to, the administration of a depot injection). In certain aspects, the term refers to the reduction or amelioration of a vitamin deficiency (including, but not limited to, vitamin B12 deficiency).

The term “vitamer” as used in the present disclosure refers to a chemical compound structurally similar to a vitamin, e.g. vitamin B12, and that exhibit similar or the same biological activity as the vitamin.

The term “osmotic agent” as used herein, refers to compounds capable of imbibing water and can thereby establish an osmotic pressure gradient across an adjacent semipermeable barrier. Osmotic agents are also referred to as “osmogens” or “osmagents”.

The term “g/g” as used herein refers to the ratio between two components with respect to mass in grams (g). For example, a composition comprising 2 g polymer and 1 g active ingredient would have a polymer to active ingredient ratio (g/g) of 2.

The term “mg/mL” as used herein refers to the ratio between a solute, generally an active pharmaceutical ingredient or excipient, and a solvent, generally but not necessarily water. For example, a 50 mg/mL sodium chloride aqueous solution would represent a solution comprising 50 mg sodium chloride for every 1 mL water.

The terms “depot”, “long acting”, and “sustained release” as used herein refer to a drug release profile in which the drug release characteristics of time course and/or location are chosen to accomplish therapeutic or convenience objectives not offered by conventional dosage forms such as solutions, suspensions, or promptly dissolving dosage forms.

The term “microparticle” as used herein refers to structures or particles having sizes from about 10 nm to 1000 μm and includes microcapsules, microspeheres, nanoparticles, nanocapsules, nanospheres, as well as particles in general that are less than about 1000 μm.

The term “microemulsion” (“ME”) as used herein refers to thermodynamically stable, transparent dispersions of water and oil, stabilized by an interfacial film of surfactant molecules. Microemulsions are characterized by their submicron particle size of 0.1 μm or below.

### Pharmaceutical Compositions

Provided herein are pharmaceutical sustained release compositions comprising vitamers of vitamin B12. In some aspects, the compositions are injectable depot pharmaceutical compositions. In some aspects, the compositions are injectable long acting pharmaceutical compositions. In some aspects, the compositions are injectable sustained release pharmaceutical compositions. In some aspects, the vitamer of vitamin B12 is selected from the group consisting of cyanocobalamin, hydroxocobalamin, adenosylcobalamin, and methylcobalamin.

In some aspects, the vitamer is a pharmaceutically acceptable salt. In some aspects, the pharmaceutically acceptable salt is an organic salt. Pharmaceutically acceptable organic salts can be prepared by admixing a vitamer of vitamin B12, e.g., cyanocobalamin, with an organic acid. In some aspects, the organic acid is pamoic acid, palmitic acid, oleic acid, octanoic acid, decanoic acid, hexanoic acid, or stearic acid. The admixing can be performed separately, whereafter the pharmaceutically acceptable salt is added to the final composition, or can be performed in situ. In some aspects, the vitamer and the organic acid are mixed in a 1:3 ratio. In some aspects, the vitamer and the organic acid are mixed in a 1:1 ratio, a 3:1 ratio, a 2:1 ratio, or a 1:2 ratio. In some aspects, the vitamer is the pamoate salt of cyanocobalamin, i.e., cyanocobalamin pamoate. In some aspects, the vitamer is an oleate salt of cyanocobalamin, an octanoate salt of cyanocobalamin, a decanoate salt of cyanocobalamin, a hexanoate salt of cyanocobalamin, a palmitate salt of cyanocobalamin or a stearate salt of cyanocobalamin.

Applicant has unexpectedly discovered that organic salts of cyanocobalamin, e.g., cyanocobalamin pamoate, dissolve significantly more effectively in compositions comprising a polymer, e.g., a polylactide, a poly(lactide-co-glycolide), or a combination thereof, and a solvent, e.g., DMSO, NMP, or a combination thereof. Such compositions may be more effective in releasing cyanocobalamin over an extended period of time, e.g., multiple weeks, relative to a composition comprising free cyanocobalamin or an inorganic salt of cyanocobalamin and the same polymer and solvent. Without wishing to be bound by theory, it is believed that compositions comprising organic salts of cyanocobalamin, e.g., cyanocobalamin pamoate, are more stable because the propensity for drug precipitation is reduced.

In some aspects, the composition comprises a biodegradable carrier. In some aspects, the biodegradable carrier is a lipid, an oil, a polymer, or a combination or mixture thereof.

In some aspects, the composition comprises a biodegradable polymer. Non-limiting examples of biodegradable polymers include polylactides, polyglycolides, poly(lactide-co-glycolide) s, polycaprolactones (e.g., poly(ε-caprolactone) (PCL)), polydioxanones, polycarbonates (e.g., (polyethylene carbonate) (PEC)), polyhydroxybutyrates, poly(3-hydroxybutyrate-co-3-hydroxyvalerate), tri (ethylene glycol) poly(orthoester), polyalkylene oxalates, polyanhydrides, polyamides, polyesteramides, polyurethanes, polyacetals, polyorthocarbonates, polyphosphazenes, polyhydroxyvalerates, polyalkylene succinates, polyorthoesters, sucrose acetate isobutyrate (SAIB), and copolymers, block copolymers, branched copolymers, terpolymers, and combinations and mixtures thereof.

In some aspects, the biodegradable polymer is a polylactide, a poly(lactide-co-glycolide), or a combination thereof. Poly(lactide-co-glycolide) is a co-polymer of lactic acid (lactide) and glycolic acid (glycolide). Exemplary poly(lactide-co-glycolide) s include those manufactured by Evonik under the brand name RESOMER®, e.g., RESOMER® RG 502 H, RESOMER® RG 503 H, and RESOMER® RG 653 H; those manufactured by Ashland under the brand name Viatel™, e.g., Viatel™ DLG 5002 A, Viatel™ DLG 5002 E, Viatel™ DLG 5003 A, Viatel™ DLG 5005 A, Viatel™ DLG 5503 A, and Viatel™ DLG 6503 A. Exemplary polylactides include those manufactured by Ashland under the brand name Viatel™, e.g., Viatel™ DL 02 A.

The poly(lactide-co-glycolide) may have any ratio of lactide to glycolide, also referred to as the LA: GA ratio. In some aspects, the ratio of lactide to glycolide is from about 5:95 to about 95:5. In some aspects, the ratio of lactide to glycolide is from about 90:10 to about 75:25, from about 90:10 to about 65:35, from about 90:10 to about 50:50, from about 90:10 to about 35:65, from about 90:10 to about 25:75, from about 90:10 to about 10:90, from about 75:25 to about 65:35, from about 75:25 to about 50:50, from about 75:25 to about 35:65, from about 75:25 to about 25:75, from about 75:25 to about 10:90, from about 65:35 to about 50:50, from about 65:35 to about 35:65, from about 65:35 to about 25:75, from about 65:35 to about 10:90, from about 50:50 to about 35:65, from about 50:50 to about 25:75, from about 50:50 to about 10:90, from about 35:65 to about 25:75, from about 35:65 to about 10:90, or from about 25:75 to about 10:90.

In some aspects, the ratio of lactide to glycolide is about 50:50. In some aspects, the ratio of lactide to glycolide is about 90:10, about 75:25, about 65:35, about 55:45, about 50:50, about 45:55, about 35:65, about 25:75, or about 10:90.

In some aspects, the biodegradable polymer has an intrinsic viscosity of from about 0.01 dL/g to about 1.0 dL/g. In some aspects, the biodegradable polymer has an intrinsic viscosity of from about 0.05 dL/g to about 0.1 dL/g, from about 0.05 dL/g to about 0.2 dL/g, from about 0.05 dL/g to about 0.3 dL/g, from about 0.05 dL/g to about 0.4 dL/g, from about 0.05 dL/g to about 0.5 dL/g, from about 0.05 dL/g to about 0.6 dL/g, from about 0.05 dL/g to about 0.7 dL/g, from about 0.05 dL/g to about 0.8 dL/g, from about 0.05 dL/g to about 0.9 dL/g, from about 0.05 dL/g to about 1 dL/g, from about 0.1 dL/g to about 0.2 dL/g, from about 0.1 dL/g to about 0.3 dL/g, from about 0.1 dL/g to about 0.4 dL/g, from about 0.1 dL/g to about 0.5 dL/g, from about 0.1 dL/g to about 0.6 dL/g, from about 0.1 dL/g to about 0.7 dL/g, from about 0.1 dL/g to about 0.8 dL/g, from about 0.1 dL/g to about 0.9 dL/g, from about 0.1 dL/g to about 1 dL/g, from about 0.2 dL/g to about 0.3 dL/g, from about 0.2 dL/g to about 0.4 dL/g, from about 0.2 dL/g to about 0.5 dL/g, from about 0.2 dL/g to about 0.6 dL/g, from about 0.2 dL/g to about 0.7 dL/g, from about 0.2 dL/g to about 0.8 dL/g, from about 0.2 dL/g to about 0.9 dL/g, from about 0.2 dL/g to about 1 dL/g, from about 0.3 dL/g to about 0.4 dL/g, from about 0.3 dL/g to about 0.5 dL/g, from about 0.3 dL/g to about 0.6 dL/g, from about 0.3 dL/g to about 0.7 dL/g, from about 0.3 dL/g to about 0.8 dL/g, from about 0.3 dL/g to about 0.9 dL/g, from about 0.3 dL/g to about 1 dL/g, from about 0.4 dL/g to about 0.5 dL/g, from about 0.4 dL/g to about 0.6 dL/g, from about 0.4 dL/g to about 0.7 dL/g, from about 0.4 dL/g to about 0.8 dL/g, from about 0.4 dL/g to about 0.9 dL/g, from about 0.4 dL/g to about 1 dL/g, from about 0.5 dL/g to about 0.6 dL/g, from about 0.5 dL/g to about 0.7 dL/g, from about 0.5 dL/g to about 0.8 dL/g, from about 0.5 dL/g to about 0.9 dL/g, from about 0.5 dL/g to about 1 dL/g, from about 0.6 dL/g to about 0.7 dL/g, from about 0.6 dL/g to about 0.8 dL/g, from about 0.6 dL/g to about 0.9 dL/g, from about 0.6 dL/g to about 1 dL/g, from about 0.7 dL/g to about 0.8 dL/g, from about 0.7 dL/g to about 0.9 dL/g, from about 0.7 dL/g to about 1 dL/g, from about 0.8 dL/g to about 0.9 dL/g, from about 0.8 dL/g to about 1 dL/g, or from about 0.9 dL/g to about 1 dL/g.

In some aspects, the biodegradable polymer has an intrinsic viscosity of about 0.25 dL/g. In some aspects, the biodegradable polymer has an intrinsic viscosity of about 0.05 dL/g, about 0.1 dL/g, about 0.15 dL/g, about 0.2 dL/g, about 0.25 dL/g, about 0.3 dL/g, about 0.35 dL/g, about 0.4 dL/g, about 0.45 dL/g, about 0.5 dL/g, about 0.55 dL/g, about 0.6 dL/g, about 0.65 dL/g, about 0.7 dL/g, about 0.75 dL/g, about 0.8 dL/g, about 0.85 dL/g, about 0.9 dL/g, about 0.95 dL/g, or about 1 dL/g.

In some aspects, the polymer has a molecular weight of from about 5 kDa to about 100 kDa. In some aspects, the polymer has a molecular weight of from about 1 kDa to about 5 kDa, from about 1 kDa to about 10 kDa, from about 1 kDa to about 15 kDa, from about 1 kDa to about 20 kDa, from about 1 kDa to about 25 kDa, from about 1 kDa to about 30 kDa, from about 1 kDa to about 35 kDa, from about 1 kDa to about 40 kDa, from about 1 kDa to about 45 kDa, from about 1 kDa to about 50 kDa, from about 1 kDa to about 55 kDa, from about 1 kDa to about 60 kDa, from about 5 kDa to about 10 kDa, from about 5 kDa to about 15 kDa, from about 5 kDa to about 20 kDa, from about 5 kDa to about 25 kDa, from about 5 kDa to about 30 kDa, from about 5 kDa to about 35 kDa, from about 5 kDa to about 40 kDa, from about 5 kDa to about 45 kDa, from about 5 kDa to about 50 kDa, from about 5 kDa to about 55 kDa, from about 10 kDa to about 15 kDa, from about 10 kDa to about 20 kDa, from about 10 kDa to about 25 kDa, from about 10 kDa to about 30 kDa, from about 10 kDa to about 35 kDa, from about 10 kDa to about 40 kDa, from about 10 kDa to about 45 kDa, from about 10 kDa to about 50 kDa, from about 10 kDa to about 55 kDa, from about 10 kDa to about 60 kDa, from about 15 kDa to about 20 kDa, from about 15 kDa to about 25 kDa, from about 15 kDa to about 30 kDa, from about 15 kDa to about 35 kDa, from about 15 kDa to about 40 kDa, from about 15 kDa to about 45 kDa, from about 15 kDa to about 50 kDa, from about 15 kDa to about 55 kDa, from about 15 kDa to about 60 kDa, from about 20 kDa to about 25 kDa, from about 20 kDa to about 30 kDa, from about 20 kDa to about 35 kDa, from about 20 kDa to about 40 kDa, from about 20 kDa to about 45 kDa, from about 20 kDa to about 50 kDa, from about 20 kDa to about 55 kDa, from about 20 kDa to about 60 kDa, from about 25 kDa to about 30 kDa, from about 25 kDa to about 35 kDa, from about 25 kDa to about 40 kDa, from about 25 kDa to about 45 kDa, from about 25 kDa to about 50 kDa, from about 25 kDa to about 55 kDa, from about 25 kDa to about 60 kDa, from about 30 kDa to about 35 kDa, from about 30 kDa to about 40 kDa, from about 30 kDa to about 45 kDa, from about 30 kDa to about 50 kDa, from about 30 kDa to about 55 kDa, from about 30 kDa to about 60 kDa, from about 35 kDa to about 40 kDa, from about 35 kDa to about 45 kDa, from about 35 kDa to about 50 kDa, from about 35 kDa to about 55 kDa, from about 35 kDa to about 60 kDa, from about 40 kDa to about 45 kDa, from about 40 kDa to about 50 kDa, from about 40 kDa to about 55 kDa, from about 40 kDa to about 60 kDa, from about 45 kDa to about 50 kDa, from about 45 kDa to about 55 kDa, from about 45 kDa to about 60 kDa, from about 50 kDa to about 55 kDa, from about 50 kDa to about 60 kDa, or from about 55 kDa to about 60 kDa.

In some aspects, the polymer has a molecular weight of about 25 kDa. In some aspects, the polymer has a molecular weight of about 1 kDa, about 5 kDa, about 10 kDa, about 15 kDa, about 20 kDa, about 25 kDa, about 30 kDa, about 35 kDa, about 40 kDa, about 45 kDa, about 50 kDa, about 55 kDa, or about 60 kDa.

In some aspects, the concentration of the polymer is from about 20% to about 40%. In some aspects, the concentration of the polymer is from about 5% to about 10%, from about 5% to about 15%, from about 5% to about 20%, from about 5% to about 25%, from about 5% to about 30%, from about 5% to about 35%, from about 5% to about 40%, from about 5% to about 45%, from about 5% to about 50%, from about 10% to about 15%, from about 10% to about 20%, from about 10% to about 25%, from about 10% to about 30%, from about 10% to about 35%, from about 10% to about 40%, from about 10% to about 45%, from about 10% to about 50%, from about 15% to about 20%, from about 15% to about 25%, from about 15% to about 30%, from about 15% to about 35%, from about 15% to about 40%, from about 15% to about 45%, from about 15% to about 50%, from about 20% to about 25%, from about 20% to about 30%, from about 20% to about 35%, from about 20% to about 45%, from about 20% to about 50%, from about 25% to about 30%, from about 25% to about 35%, from about 25% to about 40%, from about 25% to about 45%, from about 25% to about 50%, from about 30% to about 35%, from about 30% to about 40%, from about 30% to about 45%, from about 30% to about 50%, from about 35% to about 40%, from about 35% to about 45%, from about 35% to about 50%, from about 40% to about 45%, from about 40% to about 50%, or from about 45% to about 50%.

In some aspects, the concentration of the polymer is about 30%. In some aspects, the concentration of the polymer is about 5%, about 10%, about 15%, about 20%, about 21%, about 22%, about 23%, about 24%, about 25%, about 26%, about 27%, about 28%, about 29%, about 31%, about 32%, about 33%, about 34%, about 35%, about 36%, about 37%, about 38%, about 39%, about 40%, about 45%, or about 50%.

In some aspects, the composition comprises an oil. Non-limiting examples of oils include medium chain triglycerides of caprylic (C8) and capric (C10) acids, any pure fatty acid ester such as ethyl, propyl isopropyl, and butyl; esters of fatty acids such as caproic, caprylic, capric, lauric, palmitic, myristic, and stearic acids, isopropyl myristate, isopropyl palmitate, isopropyl caproate, isopropyl caprylate, ethyl stearate, butyl laurate, coconut oil, palm kernel oil, soy bean oil, castor oil, cotton seed oil, corn oil, and olive oil, and combinations and mixtures thereof.

In some aspects, the composition comprises a lipid. Non-limiting examples of lipids include phosphatidylcholines, phoshatidylethanolamines, phosphatidic acids, phosphatidylinositols, phosphatidylserines, phosphatidylglycerols, diphosphatidylglycerols, sterols, lecithins, and combinations and mixtures thereof.

In some aspects, the composition is stable for at least 6 months at 25° C. In some aspects, the composition is stable for at least 9 months at 25° C.

In some aspects, the composition is stable for at least 12 months at 25° C.

In some aspects, the composition is stable for at least 18 months at 25° C.

In some aspects, the composition is lyophilized into a solid powder.

In some aspects, the amount of vitamer of vitamin B12 present is from about 0.1 mg to about 0.5 mg, from about 0.1 mg to about 0.7 mg, from about 0.1 mg to about 1 mg, from about 0.1 mg to about 3 mg, from about 0.1 mg to about 5 mg, from about 0.5 mg to about 0.7 mg, from about 0.5 mg to about 1 mg, from about 0.5 mg to about 3 mg, from about 0.5 mg to about 5 mg, from about 0.7 mg to about 1 mg, from about 0.7 mg to about 3 mg, from about 0.7 mg to about 5 mg, from about 1 mg to about 3 mg, from about 1 to about 5 mg, or from about 3 to about 5 mg. In some aspects, the vitamer is cyanocobalamin.

In some aspects, the amount of vitamer of vitamin B12 present is about 0.1 mg, about 0.5 mg, about 0.7 mg, about 0.8 mg, about 1 mg, about 2 mg, about 3 mg, about 4 mg, or about 5 mg.

In some aspects, the concentration of the vitamer of vitamin B12 is from about 1 mg/g to about 6 mg/g (mg of vitamer relative to total mass of composition in grams). In some aspects, the concentration of the vitamer of vitamin B12 is from about 1 mg/g to about 2 mg/g, from about 1 mg/g to about 3 mg/g, from about 1 mg/g to about 4 mg/g, from about 1 mg/g to about 5 mg/g, from about 1 mg/g to about 7 mg/g, from about 1 mg/g to about 8 mg/g, from about 1 mg/g to about 9 mg/g, from about 1 mg/g to about 10 mg/g, from about 2 mg/g to about 3 mg/g, from about 2 mg/g to about 4 mg/g, from about 2 mg/g to about 5 mg/g, from about 2 mg/g to about 6 mg/g, from about 2 mg/g to about 7 mg/g, from about 2 mg/g to about 8 mg/g, from about 2 mg/g to about 9 mg/g, from about 2 mg/g to about 10 mg/g, from about 3 mg/g to about 4 mg/g, from about 3 mg/g to about 5 mg/g, from about 3 mg/g to about 6 mg/g, from about 3 mg/g to about 7 mg/g, from about 3 mg/g to about 8 mg/g, from about 3 mg/g to about 9 mg/g, from about 3 mg/g to about 10 mg/g, from about 4 mg/g to about 5 mg/g, from about 4 mg/g to about 6 mg/g, from about 4 mg/g to about 7 mg/g, from about 4 mg/g to about 8 mg/g, from about 4 mg/g to about 9 mg/g, from about 4 mg/g to about 10 mg/g, from about 5 mg/g to about 6 mg/g, from about 5 mg/g to about 7 mg/g, from about 5 mg/g to about 8 mg/g, from about 5 mg/g to about 9 mg/g, from about 5 mg/g to about 10 mg/g, from about 6 mg/g to about 7 mg/g, from about 6 mg/g to about 8 mg/g, from about 6 mg/g to about 9 mg/g, from about 6 mg/g to about 10 mg/g, from about 7 mg/g to about 8 mg/g, from about 7 mg/g to about 9 mg/g, from about 7 mg/g to about 10 mg/g, from about 8 mg/g to about 9 mg/g, from about 8 mg/g to about 10 mg/g, or from about 9 mg/g to about 10 mg/g.

In some aspects, the concentration of the vitamer of vitamin B12 is about 4 mg/g. In some aspects, the concentration of the vitamer of vitamin B12 is about 0.5 mg/g, about 1 mg/g, about 1.5 mg/g, about 2 mg/g, about 2.5 mg/g, about 3 mg/g, about 3.5 mg/g, about 4 mg/g, about 4.5 mg/g, about 5 mg/g, about 5.5 mg/g, about 6 mg/g, about 6.5 mg/g, about 7 mg/g, about 7.5 mg/g, about 8 mg/g, about 8.5 mg/g, about 9 mg/g, about 9.5 mg/g, or about 10 mg/g.

In some aspects, the composition has a pH of from about 5 to about 8. In some aspects, the composition has a pH of from about 5 to about 5.5, from about 5 to about 6, from about 5 to about 6.5, from about 5 to about 7, from about 5 to about 7.5, from about 5 to about 8, from about 5.5 to about 6, from about 5.5 to about 6.5, from about 5.5 to about 7, from about 5.5 to about 7.5, from about 5.5 to about 8, from about 6 to about 6.5, from about 6 to about 7, from about 6 to about 7.5, from about 6 to about 8, from about 6.5 to about 7, from about 6.5 to about 7.5, from about 6.5 to about 8, from about 7 to about 7.5, or from about 7.5 to about 8.

In some aspects, the composition has a pH of about 5, about 5.5, about 6, about 6.5, about 7, about 7.5, or about 8.

In some aspects, the pharmaceutical composition comprises one or more pharmaceutically acceptable excipients. In some aspects, the excipient is a preservative, a buffer, a bulking agent, a wetting agent, a tonicity agent, a sugar, a lubricant, an emollient, an antioxidant, a solvent, a surfactant, a stabilizer, a salt, an osmotic agent, a chelating agent, a resuspension component, a polymer, a lipid, or a combination of excipients thereof. The excipient can serve various purposes. A person skilled in the art can select one or more excipients with respect to the particular desired properties by routine experimentation and without any undue burden. The amount of each excipient used can vary within ranges conventional in the art. Techniques and excipients which can be used to formulate dosage forms are described in Handbook of Pharmaceutical Excipients, 6th edition, Rowe et al., Eds., American Pharmaceuticals Association and the Pharmaceutical Press, publications department of the Royal Pharmaceutical Society of Great Britain (2009); and Remington: the Science and Practice of Pharmacy, 21th edition, Gennaro, Ed., Lippincott Williams & Wilkins (2005).

In some aspects, the pharmaceutical composition comprises a solvent. In some aspects, the solvent is an organic solvent. In some aspects, the organic solvent is a water-miscible solvent. Non-limiting examples of water-miscible solvents include dimethyl sulfoxide (DMSO), N-methyl-2-pyrrolidone (NMP), triacetin, propylene glycol, and combinations thereof. In some aspects, the water-miscible solvent is DMSO. In some aspects, the organic solvent is a water-immiscible solvent. Non-limiting examples of water-immiscible solvents include benzyl alcohol, benzyl benzoate, and combinations thereof.

In some aspects, the pharmaceutical composition comprises one or more buffers in an amount effective to control and/or maintain the pH of the composition. The buffer may be chosen from those that are conventional and well known in the art. Non-limiting examples of such buffers include acetate buffers, citrate buffers, phosphate buffers, borate buffers, borate-polyol buffers, carbonate buffers, organic buffers, amino acid buffers, TRIS buffers, and combinations thereof. The amount of buffer employed preferably is sufficient to maintain the pH of the composition in a range of from about 5 to about 5.5, from about 5 to about 6, from about 5 to about 6.5, from about 5 to about 7, from about 5 to about 7.5, from about 5 to about 8, from about 5.5 to about 6, from about 5.5 to about 6.5, from about 5.5 to about 7, from about 5.5 to about 7.5, from about 5.5 to about 8, from about 6 to about 6.5, from about 6 to about 7, from about 6 to about 7.5, from about 6 to about 8, from about 6.5 to about 7, from about 6.5 to about 7.5, from about 6.5 to about 8, from about 7 to about 7.5, or from about 7.5 to about 8.

In some aspects, the pharmaceutical composition comprises an osmotic agent. Non-limiting examples of osmotic agents include glycerin, sodium chloride, magnesium chloride, calcium chloride, potassium chloride, magnesium sulfate, potassium sulfate, sodium carbonate, sodium sulfate, ascorbic acid, benzoic acid, fumaric acid, citric acid, mannitol, sucrose, sorbitol, xylitol, lactose, dextrose, and trehalose.

In some aspects, the osmotic agent is present at a concentration of from about 0.1 mg/mL to about 1 mg/mL, from about 0.1 mg/mL to about 5 mg/mL, from about 0.1 mg/mL to about 10 mg/mL, from about 0.1 mg/mL to about 20 mg/mL, from about 1 mg/mL to about 5 mg/mL, from about 1 mg/mL to about 10 mg/mL, from about 1 mg/mL to about 20 mg/mL, from about 5 mg/mL to about 10 mg/mL, from about 5 mg/mL to about 20 mg/mL, or from about 10 mg/mL to about 20 mg/mL. In some aspects, the osmotic agent is present at a concentration of about 0.1 mg/mL, about 1 mg/mL, about 5 mg/mL, about 10 mg/mL, or about 20 mg/mL.

In some aspects, the composition comprises a preservative. Non-limiting examples of preservatives include o-cresol, m-cresol, and p-cresol, benzyl alcohol, and parabens such methylparaben and propylparaben. In some aspects, the preservative is present at a concentration of about 5 mg/mL to about 100 mg/mL.

In some aspects, composition comprises an antioxidant. Non-limiting examples of antioxidants include methionine, monothioglycerol, sodium bisulfite, sodium metabisulfite, sodium thiosulfate, sodium citrate, sodium ascorbate, and thiourea, or combinations thereof.

In some aspects, the composition comprises a chelating agent. In one aspect, the chelating agent is disodium edetate (EDTA) or pentetic acid (DTPA), but others can be used instead of or in combination with this.

In some aspects, the composition is formulated for parenteral administration.

In some aspects, the compositions disclosed herein slowly release the vitamer of vitamin B12 over an extended period of time, i.e., when the composition is administered to a subject, the vitamer of vitamin B12 becomes bioavailable in the subject over an extended period of time, e.g., 7 days, 14 days, 21 days, or longer. In some aspects, from about 30% to about 60% of the vitamer of vitamin B12 is released after about 7 days. In some aspects, from about 10% to about 20%, from about 10% to about 30%, from about 10% to about 40%, from about 10% to about 50%, from about 10% to about 60%, from about 10% to about 70%, from about 20% to about 30%, from about 20% to about 40%, from about 20% to about 50%, from about 20% to about 60%, from about 20% to about 70%, from about 30% to about 40%, from about 30% to about 50%, from about 30% to about 70%, from about 40% to about 50%, from about 40% to about 60%, from about 40% to about 70%, from about 50% to about 60%, from about 50% to about 70%, or from about 60% to about 70% of the vitamer of vitamin B12 is released after about 7 days.

In some aspects, from about 50% to about 80% of the vitamer of vitamin B12 is released after about 14 days. In some aspects, from about 10% to about 20%, from about 10% to about 30%, from about 10% to about 40%, from about 10% to about 50%, from about 10% to about 60%, from about 10% to about 70%, from about 10% to about 80%, from about 20% to about 30%, from about 20% to about 40%, from about 20% to about 50%, from about 20% to about 60%, from about 20% to about 70%, from about 20% to about 80%, from about 30% to about 40%, from about 30% to about 50%, from about 30% to about 60%, from about 30% to about 70%, from about 30% to about 80%, from about 40% to about 50%, from about 40% to about 60%, from about 40% to about 70%, from about 40% to about 80%, from about 50% to about 60%, from about 50% to about 70%, from about 60% to about 70%, from about 60% to about 80%, or from about 70% to about 80% of the vitamer of vitamin B12 is released after about 14 days.

In some aspects, the compositions disclosed herein initially release the vitamer of vitamin B12 in a first “burst phase” lasting about 1 or 2 days; then release additional vitamer of vitamin B12 at a slower rate in a “slow release phase” lasting from about 3 to about 9 days; then release additional vitamer of vitamin B12 at a faster rate in a second burst phase lasting about 1 or 2 days.

In some aspects, the composition is dispersed in an oil. Non-limiting examples of oils are listed above.

In some aspects, the composition comprises microparticles. Microparticles include both microspheres and microcapsules, and may be approximately spherical or have other geometries. Microspheres are typically approximately homogeneous in composition and microcapsules comprise a core of a composition distinct from a surrounding shell. Microparticles may aggregate into larger masses under some circumstances. The microparticle will generally be of a diameter that permits parenteral administration without occluding needles and capillaries. Microparticle size is readily determined by techniques well known in the art, such as photon correlation spectroscopy, laser diffractometry and/or scanning electron microscopy. Polymer microparticles for use herein are typically formed from materials that are sterilizable, substantially non-toxic, and biodegradable.

In some aspects, the microparticles comprise a vitamer of vitamin B12. In some aspects, the vitamer is selected from the group consisting of cyanocobalamin, hydroxocobalamin, adenosylcobalamin, and methylcobalamin. In some aspects, the vitamer is cyanocobalamin.

In some aspects, the microparticle has a diameter less than 1000 μm. In some aspects, the microparticle has a diameter of from about 0.5 μm to about 5 μm, from about 0.5 μm to about 10 μm, from about 0.5 μm to about 25 μm, from about 0.5 μm to about 50 μm, from about 0.5 μm to about 75 μm, from about 0.5 μm to about 100 μm, from about 0.5 μm to about 500 μm, from about 0.5 μm to about 1000 μm, from about 10 μm to about 25 μm, from about 10 μm to about 50 μm, from about 10 μm to about 75 μm, from about 10 μm to about 100 μm, from about 10 μm to about 500 μm, from about 10 μm to about 1000 μm, from about 25 μm to about 50 μm, from about 25 μm to about 75 μm, from about 25 μm to about 100 μm, from about 25 μm to about 500 μm, from about 25 μm to about 1000 μm, from about 50 μm to about 75 μm, from about 50 μm to about 100 μm, from about 50 μm to about 500 μm, from about 50 μm to about 1000 μm, from about 75 μm to about 100 μm, from about 75 μm to about 500 μm, from about 75 μm to about 1000 μm, from about 100 μm to about 500 μm, from about 100 μm to about 1000 μm, or from about 500 μm to about 1000 μm. In some aspects, the microparticle has a diameter of about 0.5 μm, about 50 μm, about 100 μm, about 200 μm, about 300 μm, about 400 μm, about 500 μm, or about 1000 μm. In some aspects, the microparticle has a diameter of about 5 μm, about 10 μm, about 25 μm, about 50 μm, or about 75 μm.

In some aspects, microparticles can be made with one or more biodegradable polymers. Non-limiting examples of biodegradable polymers are listed above.

In some aspects, the molecular weight of the biodegradable polymer is from about 5 kDa to about 10 kDa, from about 5 kDa to about 15 kDa, from about 5 kDa to about 20 kDa, from about 5 kDa to about 25 kDa, from about 5 kDa to about 50 kDa, from about 5 kDa to about 100 kDa, from about 5 kDa to about 200 kDa, from about 5 kDa to about 300 kDa, from about 10 kDa to about 15 kDa, from about 10 kDa to about 20 kDa, from about 10 kDa to about 25 kDa, from about 10 kDa to about 50 kDa, from about 10 kDa to about 100 kDa, from about 10 kDa to about 200 kDa, from about 10 kDa to about 300 kDa, from about 15 kDa to about 20 kDa, from about 15 kDa to about 25 kDa, from about 15 kDa to about 50 kDa, from about 15 kDa to about 100 kDa, from about 15 kDa to about 200 kDa, from about 15 kDa to about 300 kDa, from about 20 kDa to about 25 kDa, from about 20 kDa to about 50 kDa, from about 20 kDa to about 100 kDa, from about 20 kDa to about 200 kDa, from about 20 kDa to about 300 kDa, from about 25 kDa to about 50 kDa, from about 25 kDa to about 100 kDa, from about 25 kDa to about 200 kDa, from about 25 kDa to about 300 kDa, from about 50 kDa to about 100 kDa, from about 50 kDa to about 200 kDa, from about 50 kDa to about 300 kDa, from about 100 kDa to about 200 kDa, from about 100 kDa to about 300 kDa, or from about 200 kDa to about 300 kDa, In some aspects the molecular weight of the biodegradable polymer is about 5 kDa, about 10 kDa, about 15 kDa, about 20 kDa, about 25 kDa, about 50 kDa, about 100 kDa, about 200 kDa, or about 300 kDa.

In some aspects, the microparticles have a monodisperse size population. In some aspects, the microparticles have a polydispersity index of from about 1.0 to about 1.5, from about 1.0 to about 2.0, from about 1.0 to about 2.5, from about 1.0 to about 3.0, from about 1.0 to about 3.5, from about 1.5 to about 2.0, from about 1.5 to about 2.5, from about 1.5 to about 3.0, from about 1.5 to about 3.5, from about 2.0 to about 2.5, from about 2.0 to about 3.0, from about 2.0 to about 3.5, from about 2.5 to about 3.0, from about 2.5 to about 3.5, or from about 3.0 to about 3.5.

In some aspects, the microparticles comprise less than 1 wt % chlorinated solvents. In some aspects, the microparticles comprise less than about 0.9 wt %, less than about 0.8 wt %, less than about 0.7 wt %, less than about 0.6 wt %, less than about 0.5 wt %, less than about 0.4 wt %, less than about 0.3 wt %, less than about 0.2 wt %, or less than about 0.1 wt % chlorinated solvents. In some aspects, the microparticles are essentially free of chlorinated solvents. In some aspects, the microparticles are synthesized without using chlorinated solvents. In some aspects, the microparticles do not comprise chlorinated solvents.

In some aspects, the ratio (g/g) of the vitamer of vitamin B12 to polymer is from about 0.01 to about 0.05, from about 0.01 to about 0.1, from about 0.01 to about 0.2, from about 0.01 to about 0.3, from about 0.05 to about 0.1, from about 0.05 to about 0.2, from about 0.05 to about 0.3, from about 0.1 to about 0.2, from about 0.1 to about 0.3, or from about 0.2 to about 0.3. In some aspects, the ratio (g/g) of the vitamer of vitamin B12 to polymer is about 0.01, about 0.05, about 0.1, about 0.2, or about 0.3.

In some aspects, the composition comprising microparticles is a depot pharmaceutical composition. In some aspects, the composition is a long acting pharmaceutical composition. In some aspects, the composition is a sustained release pharmaceutical composition.

In some aspects, the composition comprises a liposome. Liposomes can be prepared as described in Liposomes: Rational Design (A. S. Janoff, ed., Marcel Dekker, Inc., New York, N.Y.), or by additional techniques known to those knowledgeable in the art. The liposomes can comprise an internal aqueous compartment. In some aspects, the liposomes are large unilamellar vesicles (LUVs), multilamellar vesicles (MLVs), small unilamellar vesicles (SUVs) and interdigitating fusion liposomes. In some aspects, the liposomes are comprised of lipids. Non-limiting examples of lipids are listed above. In some aspects, the liposomes comprise a surface stabilizing hydrophilic polymer-lipid conjugate. In some aspects, the surface stabilizing hydrophilic polymer-lipid conjugate comprises polyethylene glycol.

In some aspects, the liposomes comprise a vitamer of vitamin B12. In some aspects, the vitamer is selected from the group consisting of cyanocobalamin, hydroxocobalamin, adenosylcobalamin, and methylcobalamin. In some aspects, the vitamer is cyanocobalamin.

In some aspects, the composition comprising liposomes is a depot pharmaceutical composition. In some aspects, the composition is a long acting pharmaceutical composition. In some aspects, the composition is a sustained release pharmaceutical composition.

In some aspects, the composition comprises a microemulsion. In the present disclosure, a microemulsion comprises: i) a discontinuous internal phase comprising an aqueous solution encompassed within an internal emulsifier; ii) an oil phase encompassing the internal phase; and iii) an external emulsifier encompassing the oil phase. In some aspects, the internal phase comprises a vitamer of vitamin B12. In some aspects, the vitamer is selected from the group consisting of cyanocobalamin, hydroxocobalamin, adenosylcobalamin, and methylcobalamin. In some aspects, the vitamer is cyanocobalamin.

In some aspects, the emulsion comprises an internal emulsifier. Non-limiting examples of internal emulsifiers include propylene glycol monocaprylate or any other surfactant with an HLB value of from about 3 to about 7 and/or propylene glycol ester of any fatty acid such as propylene glycol monocaproate, propylene glycol monocaprylate, propylene glycol monocaprate, propylene glycol monolaurate, propylene glycol monostearate, propylene glycol monopalmitate, polyethylene glycol lauryl ether, polyethylene glycol oleyl ether, polyethylene glycol hexadecyl ether, sorbitan monopalmitate, sorbitan monostearate, sorbitan monooleate, sorbitan monolaurate, transcutol P, gelucire 50/13, gelucire 44/14, gelucire 43/01, any PEG mono-, di- and/or tri-esters of any fatty acid, lecithin, egg lecithin, phosphatidylcholine, phosphatidylethanolamine, phosphatidylinositol, tocopherol or any other phospholipid, tween 80, and combinations thereof.

In some aspects, the emulsion comprises an oil phase. Non-limiting examples of oils are listed above.

In some aspects, the emulsion comprises an external emulsifier. Non-limiting examples of external emulsifiers include caprylo-caproyl polyoxyl-8 glycerides, macrogolglycerol ricinoleate, any other hydrophilic surfactant with a Hydrophile Lipophile Balance (HLB) value of from about 10 to about 16, polyethylene glycol mono- and/or di-esters of any fatty acid or fatty acid mixture, propylene glycol or any other alcohol including but not limited to glycerol, polyethylene glycol, ethanol, propanol, and isopropanol, and combinations thereof.

In some aspects, the composition comprising emulsions is a depot pharmaceutical composition. In some aspects, the composition is a long acting pharmaceutical composition. In some aspects, the composition is a sustained release pharmaceutical composition.

In some aspects, the composition comprises a vitamer of vitamin B12 is chemically modified with a hydrophobic functional group. In some aspects, the aqueous solubility of the modified vitamer is decreased relative to the unmodified vitamer of vitamin B12. In some aspects, the vitamer is selected from the group consisting of cyanocobalamin, hydroxocobalamin, adenosylcobalamin, and methylcobalamin. In some aspects, the vitamer is cyanocobalamin. In some aspects, the hydrophobic functional group is an ester. In some aspects, the modified vitamer of vitamin B12 is dispersed in an oil.

In some aspects, the composition comprising a vitamer of vitamin B12 chemically modified with a hydrophobic functional group is a depot pharmaceutical composition. In some aspects, the composition is a long acting pharmaceutical composition. In some aspects, the composition is a sustained release pharmaceutical composition.

In some aspects, the composition comprises a vitamer of vitamin B12, or a pharmaceutically acceptable salt thereof, a polymer, and a solvent.

In some aspects, the composition comprises a pharmaceutically acceptable salt of a vitamer of vitamin B12, a biodegradable polymer, and a solvent.

In some aspects, the composition comprises a pharmaceutically acceptable salt of cyanocobalamin, a biodegradable polymer, and a solvent.

In some aspects, the composition comprises an organic salt of a vitamer of vitamin B12, a polymer, and a solvent.

In some aspects, the composition comprises an organic salt of a vitamer of vitamin B12, a biodegradable polymer, and a solvent.

In some aspects, the composition comprises an organic salt of a vitamer of vitamin B12, a biodegradable polymer, and a solvent.

In some aspects, the composition comprises a vitamer of vitamin B12, or a pharmaceutically acceptable salt thereof, a polymer, and an organic solvent.

In some aspects, the composition comprises a pharmaceutically acceptable salt of a vitamer of vitamin B12, a biodegradable polymer, and an organic solvent.

In some aspects, the composition comprises a pharmaceutically acceptable salt of cyanocobalamin, a biodegradable polymer, and an organic solvent.

In some aspects, the composition comprises an organic salt of a vitamer of vitamin B12, a polymer, and an organic solvent.

In some aspects, the composition comprises an organic salt of a vitamer of vitamin B12, a biodegradable polymer, and an organic solvent.

In some aspects, the composition comprises an organic salt of a vitamer of vitamin B12, a biodegradable polymer, and a solvent.

In some aspects, the composition comprises cyanocobalamin pamoate, a biodegradable polymer, and a solvent.

In some aspects, the composition comprises cyanocobalamin pamoate, a polylactide, a poly(lactide-co-glycolide), or a combination thereof, and a solvent.

In some aspects, the composition comprises cyanocobalamin pamoate, a polylactide, a poly(lactide-co-glycolide), or a combination thereof, and an organic solvent.

In some aspects, the composition comprises cyanocobalamin pamoate, a polylactide, a poly(lactide-co-glycolide), or a combination thereof, and DMSO.

In some aspects, the composition comprises cyanocobalamin pamoate, a polylactide, a poly(lactide-co-glycolide), or a combination thereof, and NMP.

### Methods of Treatment

The present disclosure also provides methods for treating vitamin B12 deficiency in a subject, the method comprising administration to a subject in need thereof of a pharmaceutical composition comprising a vitamer of vitamin B12. In some aspects, the vitamer is selected from the group consisting of cyanocobalamin, hydroxocobalamin, adenosylcobalamin, and methylcobalamin. In some aspects, the vitamer is cyanocobalamin. In some aspects, the composition is a long acting injectable depot. In some aspects, the composition is a sustained release injectable composition. In some aspects, the composition is a long acting injectable composition. In some aspects, the composition is a microparticle composition. In some aspects, the composition is a liposome composition. In some aspects, the composition comprises a vitamer of vitamin B12 chemically modified with a hydrophobic functional group.

In some aspects, the vitamin B12 deficiency is a result of Addisonian (pernicious) anemia, gastrointestinal pathology, gastrointestinal dysfunction, or gastrointestinal surgery, fish tapeworm infestation, malignancy of pancreas or bowel, folic acid deficiency, a vegetarian diet, or a vegan diet, or a combination of any of these conditions.

In some aspects, the method of administration is parenteral. In some aspects, the administration is intramuscular. In some aspects, the administration is subcutaneous.

In some aspects, the composition is administered once every 1 to 2 months, every 1 to 3 months, every 1 to 4 months, every 1 to 5 months, every 1 to 6 months, every 2 to 3 months, every 2 to 4 months, every 2 to 5 months, every 2 to 6 months, every 3 to 4 months, every 3 to 5 months, every 3 to 6 months, every 4 to 5 months, every 4 to 6 months, or every 5 to 6 months. In some aspects, the composition is administered once every month, every 2 months, every 3 months, every 4 months, every 5 months, or every 6 months. In some aspects, the composition is administered once every 0.5 to 1 months, every 0.5 to 2 months, every 0.5 to 3 months, every 0.5 to 4 months, every 0.5 to 5 months, or every 0.5 to 6 months.

In some aspects, less than about 75% of the vitamer of vitamin B12 administered to the subject is excreted in urine. In some aspects, less than about 65%, about 60%, about 50%, 40%, about 30%, about 20%, about 10%, or about 5% of the vitamer is excreted in urine.

In some aspects, the composition provides a therapeutic dose of the vitamer of vitamin B12 for at least about 4 weeks, about 8 weeks, about 12 weeks, about 16 weeks, about 20 weeks, or about 24 weeks.

In some aspects, the vitamin B12 levels in the bloodstream of a subject remain at a therapeutically effective level over a period of 2 to 6 months, wherein the therapeutically effective level is at least 160 μg/mL. In some aspects, the therapeutically effective level is at least 190 μg/mL, at least 220 μg/mL, at least 240 μg/mL, at least 260 μg/mL, or at least 280 μg/mL. In some aspects, the levels remain at therapeutically effective levels for 1 to 2 months, 1 to 3 months, 1 to 4 months, 1 to 5 months, 1 to 6 months, 2 to 3 months, 2 to 4 months, 2 to 5 months, 3 to 4 months, 3 to 5 months, 3 to 6 months, 4 to 5 months, 4 to 6 months, or 5 to 6 months.

### EXAMPLES

### Example 1

### Preparation of Microparticle Composition Comprising Cyanocobalamin

Exemplary microparticle compositions can be prepared via an oil-in-water solvent extraction/evaporation method. A non-limiting example of a microparticle composition preparation using this method is described herein. Cyanocobalamin and PLGA are dissolved in methylene chloride and an emulsion is formed according to the process disclosed in PCT publication No. WO 2005/003180, which is incorporated herein by reference in its entirety. Following solvent extraction from the emulsion particles, the hardened microparticles are sieved through a 45 μm screen. Microparticles≤45 μm are collected by centrifugation and dried by lyophilization.

### Example 2

### Preparation of Liposome Composition Comprising Cyanocobalamin

Exemplary liposomes comprising cyanocobalamin can be formulated using the following protocol. A phospholipid, 1,2-dimyristoyl-sn-glycero-3-phosphocholine (dimyristoylphosphocholine; DMPC) can be used for the liposomal composition. The phospholipid is solubilized by dissolving 200 mg of DMPC in 10 mL of t-butanol and heating the mixture in a 37° C. water bath for 5 minutes. The solution is stored at −20° C. in a container that protects the solution from exposure to light. Cyanocobalamin is solubilized by dissolving it in DMSO to a final concentration of 50 mg/mL.

To combine the phospholipid and cyanocobalamin solutions, 10 mL of DMPC in t-butanol, 0.4 ml cyanocobalamin in DMSO and 90 mL of t-butanol are mixed very well and aliquoted into small sterile glass vials containing 2.5 mL of solution each. The vials of solution are frozen in a dry ice-acetone bath and lyophilized overnight. The dried lipid mixtures are stored at −20° C.

### Example 3

### Preparation of Microemulsion (ME) Composition Comprising Cyanocobalamin

TABLE 1
Components of w/o/w microemulsion

| w/o/w microemulsion components | Quantity (g) |
|---|---|
| Surfactant | 0.7-0.9 |
| Lipid | 0.1-0.3 |
| Nonionic surfactant | 2.0-2.5 |
| Cyanocobalamin | 0.005-0.010 |
| Buffer | 0.3-0.5 |
| Water | 21-24 |
| Ethanol | 1.5-3.5 |

Exemplary microemulsions can be prepared using the following protocol. A w/o microemulsion can be prepared by mixing, under magnetic stirring at 25° C., a surfactant/lipid solution as an oily phase, a buffer solution containing dissolved cyanocobalamin as an aqueous phase, and a nonionic surfactant such as Tween 80.

The w/o microemulsion is added under stirring (at around 600 rpm for about 100 min) to a water/ethanol/Tween 80 mixture (in a ratio of approximately 15:1.5:1.0) in a microemulsion/(water/ethanol/nonionic surfactant) ratio of about 0.09:1 to give the w/o/w double microemulsion containing cyanocobalamin.

### Example 4

Preparation of Compositions Comprising a Polymer and Cyanocobalamin with or without Pamoic Acid

Compositions comprising 25% polymer and cyanocobalamin either with or without pamoic acid (i.e., to form a cyanocobalamin pamoate salt) in 100% DMSO were prepared starting from 12 mg/g stock solutions, as shown in Tables 2 and 3.

TABLE 2

| Stock Solutions (12 mg/g) | Prep 1 | Prep 2 |
|---|---|---|
| With Pamoic acid (1:3) | 11.76 | 11.89 |
| Without Pamoic acid | 11.73 | 11.85 |

TABLE 3

| | | | | Actual | |
|---|---|---|---|---|---|
| | | | Theoretical | Cyanocobalamin | |
| | | Stock | Cyanocobalamin | Concentration | |
| Composition | Polymer | Solution | Concentration | (Average of 2 | |
| Number | Used | Used | (mg/g) | preparations) | Appearance |
| 1 | Evonik | Without | 4 | 1.25 | Precipitated |
| | Resomer RG | Pamoic acid | | | solution |
| | 502 H | | | | |
| | (LA: GA, | | | | |
| | 50:50), Low | | | | |
| | Viscosity | | | | |
| 2 | Evonik | Without | 3.5 | 1.01 | Precipitated |
| | Resomer RG | Pamoic acid | | | solution |
| | 502 H | | | | |
| | (LA: GA, | | | | |
| | 50:50), Low | | | | |
| | Viscosity | | | | |
| 3 | Evonik | Without | 3 | 1.27 | Precipitated |
| | Resomer RG | Pamoic acid | | | solution |
| | 502 H | | | | |
| | (LA: GA, | | | | |
| | 50:50), Low | | | | |
| | Viscosity | | | | |
| 4 | Evonik | With | 4 | 4.07 | Clear solution |
| | Resomer RG | Pamoic acid | | | |
| | 502 H | | | | |
| | (LA: GA, | | | | |
| | 50:50), Low | | | | |
| | Viscosity | | | | |
| 5 | Evonik | With | 3.5 | 3.59 | Clear solution |
| | Resomer RG | Pamoic acid | | | |
| | 502 H | | | | |
| | (LA: GA, | | | | |
| | 50:50), Low | | | | |
| | Viscosity | | | | |
| 6 | Evonik | With | 3 | 3.08 | Clear solution |
| | Resomer RG | Pamoic acid | | | |
| | 502 H | | | | |
| | (LA: GA, | | | | |
| | 50:50), Low | | | | |
| | Viscosity | | | | |
| 7 | Evonik | Without | 4 | 0.79 | Precipitated |
| | Resomer RG | Pamoic acid | | | solution |
| | 503 H | | | | |
| | (LA: GA, | | | | |
| | 50:50), High | | | | |
| | Viscosity | | | | |
| 8 | Evonik | Without | 3.5 | 0.91 | Precipitated |
| | Resomer RG | Pamoic acid | | | solution |
| | 503 H | | | | |
| | (LA: GA, | | | | |
| | 50:50), High | | | | |
| | Viscosity | | | | |
| 9 | Evonik | Without | 3 | Sample could not | Precipitated |
| | Resomer RG | Pamoic acid | | be filtered | solution |
| | 503 H | | | | |
| | (LA: GA, | | | | |
| | 50:50), High | | | | |
| | Viscosity | | | | |
| 10 | Evonik | With | 4 | 4.02 | Clear solution |
| | Resomer RG | Pamoic acid | | | |
| | 503 H | | | | |
| | (LA: GA, | | | | |
| | 50:50), High | | | | |
| | Viscosity | | | | |
| 11 | Evonik | With | 3.5 | 3.60 | Clear solution |
| | Resomer RG | Pamoic acid | | | |
| | 503 H | | | | |
| | (LA: GA, | | | | |
| | 50:50), High | | | | |
| | Viscosity | | | | |
| 12 | Evonik | With | 3 | 3.05 | Clear solution |
| | Resomer RG | Pamoic acid | | | |
| | 503 H | | | | |
| | (LA: GA, | | | | |
| | 50:50), High | | | | |
| | Viscosity | | | | |
| 13 | Evonik 653 H | Without | 4 | 0.86 | Precipitated |
| | (LA: GA, | Pamoic acid | | | solution |
| | 65:35) | | | | |
| 14 | Evonik 653 H | Without | 2 | 1.21 | Precipitated |
| | (LA: GA, | Pamoic acid | | | solution |
| | 65:35) | | | | |
| 15 | Evonik 653 H | With | 4 | 4.10 | Clear solution |
| | (LA: GA, | Pamoic acid | | | |
| | 65:35) | | | | |
| 16 | Evonik 653 H | With | 3 | 3.10 | Clear solution |
| | (LA: GA, | Pamoic acid | | | |
| | 65:35) | | | | |
| 20 | Evonik 653 H | With | 4 | 3.98 | Clear solution |
| | (LA: GA, | Pamoic acid | | | |
| | 65:35) | | | | |
| 24 | Viatel 5002 | With | 4 | 4.00 | Clear solution |
| | (LA: GA, | Pamoic acid | | | |
| | 50:50) | | | | |
| 26 | Viatel 5003 | With | 4 | 4.1* | Clear solution |
| | (LA: GA, | Pamoic acid | | | |
| | 50:50) | | | | |
| 27 | Viatel 5503 | With | 4 | 4.09* | Clear solution |
| | (LA: GA, | Pamoic acid | | | |
| | 55:45) | | | | |
| 28 | Viatel 6503 | With | 4 | 4.12* | Clear solution |
| | (LA: GA, | Pamoic acid | | | |
| | 65:35) | | | | |
| 32 | Viatel 5002 E | With | 4 | 4.06* | Clear solution |
| | (LA: GA, | Pamoic acid | | | |
| | 50:50) | | | | |

*Single preparation

Applicant unexpectedly discovered that cyanocobalamin dissolves significantly more effectively in compositions comprising a poly(lactide-co-glycolide) and DMSO when pamoic acid is present, i.e., to form the cyanocobalamin pamoate salt, relative to when pamoic acid is not present.

### Example 5

### Preparation of Compositions Comprising a Polymer and Cyanocobalamin Pamoate

Compositions comprising polymer and cyanocobalamin pamoate in 100% DMSO were prepared as shown in Table 4.

TABLE 4

| | | | | Actual | |
|---|---|---|---|---|---|
| | | | Theoretical | Cyanocobalamin | |
| | | | Cyanocobalamin | Concentration | |
| Composition | Polymer | Polymer | Concentration | (Average of 2 | |
| Number | Used | Concentration | (mg/g) | analysis) | Appearance |
| 17 | Viatel | 30% | 4 | 4.15 | Clear |
| | 5003 | | | | Solution |
| | (LA: GA, | | | | |
| | 50:50), | | | | |
| | Low | | | | |
| | Viscosity | | | | |
| 30 | Viatel | 40% | 4 | N/A | Precipitated |
| | 5003 | | | | Solution |
| | (LA: GA, | | | | |
| | 50:50), | | | | |
| | Low | | | | |
| | Viscosity | | | | |
| 31 | Viatel | 50% | 4 | N/A | Precipitated |
| | 5003 | | | | Solution |
| | (LA: GA, | | | | |
| | 50:50), | | | | |
| | Low | | | | |
| | Viscosity | | | | |
| 25 | Viatel | 30% | 4 | 3.95 | Clear |
| | 5002 | | | | Solution |
| | (LA: GA, | | | | |
| | 50:50), | | | | |
| | High | | | | |
| | Viscosity | | | | |
| 29 | Viatel | 30% | 4 | N/A | Precipitated |
| | 5002 E | | | | Solution |
| | (LA: GA, | | | | |
| | 50:50) | | | | |
| 33 | Viatel | 30% | 4 | N/A | Precipitated |
| | 5003 | | | | Solution |
| | (LA: GA, | | | | |
| | 50:50) | | | | |
| 18 | Evonik | 30% | 4 | N/A | Precipitated |
| | Resomer | | | | Solution |
| | RG 503 H | | | | |
| | (LA: GA, | | | | |
| | 50:50), | | | | |
| | High | | | | |
| | Viscosity | | | | |
| 19 | Evonik 653 | 30% | 4 | N/A | Precipitated |
| | H | | | | Solution |
| | (LA: GA, | | | | |
| | 65:35), | | | | |
| | High | | | | |
| | Viscosity | | | | |
| 34 | Viatel | 30% | 4 | 4.11* | Clear |
| | 6503 | | | | |
| | (LA: GA, | | | | |
| | 65:35) | | | | |
| 35 | DL 02 | 30% | 4 | 4.09* | Clear |
| | (LA: GA, | | | | |
| | 100:0) | | | | |
| 33 | Viatel | 30% | 4 | 4.09* | Clear |
| | 5003 | | | | |
| | (LA: GA, | | | | |
| | 50:50) | | | | |

*Single preparation

As in Example 4, Applicant unexpectedly discovered that cyanocobalamin dissolves significantly more effectively in compositions comprising a poly(lactide-co-glycolide) and DMSO when pamoic acid is present, i.e., to form the cyanocobalamin pamoate salt, relative to when pamoic acid is not present.

### Example 6

### Preparation of Compositions Comprising a Polymer and Cyanocobalamin Pamoate

Compositions prepared in Examples 4 and 5 were tested to determine their rate of release of cyanocobalamin. Select compositions were added to buffered solutions, as shown in Table 5.

TABLE 5

| | | | | Amount | | |
|---|---|---|---|---|---|---|
| | Solu- | | | of | Solu- | |
| Com- | tion | | Grams | Com- | tion | μg of |
| position | Num- | Polymer | of | position | conc. | Cyano- |
| Number | ber | name | Buffer | added | (mg/g) | cobalamin |
| 4 | 1 | Evonik | 51.9 | 241.4 | 4.07 | 981.89 |
| | | Resomer RG | | | | |
| | | 502 H | | | | |
| | | (LA:GA, | | | | |
| | | 50:50), Low | | | | |
| | | Viscosity | | | | |
| 4 | 2 | Evonik | 50 | 249.4 | 4.07 | 1014.48 |
| | | Resomer RG | | | | |
| | | 502 H | | | | |
| | | (LA:GA, | | | | |
| | | 50:50), Low | | | | |
| | | Viscosity | | | | |
| 1 | 3 | Evonik | 50 | 258.7 | 4.02 | 1040.47 |
| | | Resomer RG | | | | |
| | | 503 H | | | | |
| | | (LA:GA, | | | | |
| | | 50:50), High | | | | |
| | | Viscosity | | | | |
| 2 | 4 | Evonik | 50.1 | 260.8 | 4.02 | 1048.63 |
| | | Resomer RG | | | | |
| | | 503 H | | | | |
| | | (LA:GA, | | | | |
| | | 50:50), High | | | | |
| | | Viscosity | | | | |
| 1 | 5 | Evonik 653 H | 50 | 251.8 | 4.10 | 1032.76 |
| | | (LA:GA, | | | | |
| | | 65:35), High | | | | |
| | | Viscosity | | | | |
| 2 | 6 | Evonik 653 H | 50 | 259.2 | 4.10 | 1063.12 |
| | | (LA:GA, | | | | |
| | | 65:35), High | | | | |
| | | Viscosity | | | | |
| 24 | 13 | Viatel 5002 | 50.1 | 255.2 | 4.00 | 1020.80 |
| | | (high | | | | |
| | | viscosity) | | | | |
| 26 | 15 | Viatel 5003 | 50 | 266.2 | 4.10 | 1091.42 |
| | | (LA:GA, | | | | |
| | | 50:50) | | | | |
| 27 | 16 | Viatel 5503 | 50 | 257.4 | 4.09 | 1052.77 |
| | | (LA:GA, | | | | |
| | | 55:45) | | | | |
| 28 | 17 | Viatel 6503 | 50.1 | 252.5 | 4.12 | 1040.30 |
| | | (LA:GA, | | | | |
| | | 65:35) | | | | |
| 20 | 18 | Evonik 653 H | 50.1 | 271.9 | 3.98 | 1082.16 |
| | | (LA:GA, | | | | |
| | | 65:35), High | | | | |
| | | Viscosity | | | | |
| DMSO | 19 | N/A | 50 | N/A | 12.085 | 982.27 |
| Stock | | | | | | |
| 2 in | | | | | | |
| Buffer | | | | | | |
| 32 | 20 | Viatel 5002 E | 50.9 | 257.1 | 4.06 | 1043.83 |
| | | (LA:GA, | | | | |
| | | 50:50) | | | | |
| 17 | 12 | Viatel 5002 | 50.1 | 254.7 | 4.15 | 1057.01 |
| | | (LA:GA, | | | | |
| | | 50:50), Low | | | | |
| | | Viscosity | | | | |
| 25 | 14 | Viatel 5002 | 50.2 | 252.7 | 3.95 | 998.17 |
| | | (LA:GA, | | | | |
| | | 50:50), High | | | | |
| | | Viscosity | | | | |
| 25 | 24 | Viatel 5002 | 50 | 252.7 | 3.95 | 998.17 |
| | | (LA:GA, | | | | |
| | | 50:50), High | | | | |
| | | Viscosity | | | | |
| 34 | 22 | Viatel 6503 | 50 | 253.1 | 4.11 | 1040.24 |
| | | (LA:GA, | | | | |
| | | 65:35) | | | | |
| 34 | 26 | Viatel 6503 | 50 | 264.7 | 4.11 | 1087.92 |
| | | (LA:GA, | | | | |
| | | 65:35) | | | | |
| 35 | 23 | DL 02 | 50 | 253.3 | 4.09 | 1036.00 |
| | | (LA:GA, | | | | |
| | | 100:0) | | | | |
| 35 | 27 | DL 02 | 50 | 255.0 | 4.09 | 1042.95 |
| | | (LA:GA, | | | | |
| | | 100:0) | | | | |
| 33 | 21 | Viatel 5003 | 50 | 252.2 | 4.09 | 1031.50 |
| | | (LA:GA, | | | | |
| | | 50:50) | | | | |
| 33 | 25 | Viatel 5003 | 50 | 253.2 | 4.09 | 1035.59 |
| | | (LA:GA, | | | | |
| | | 50:50) | | | | |

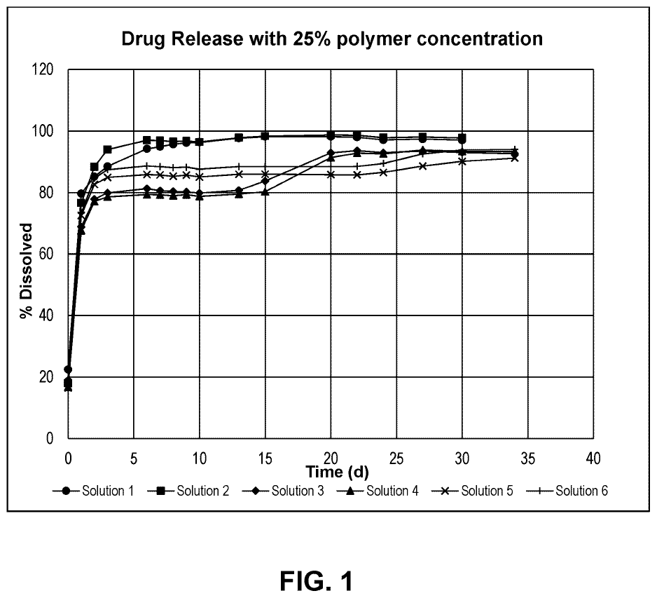

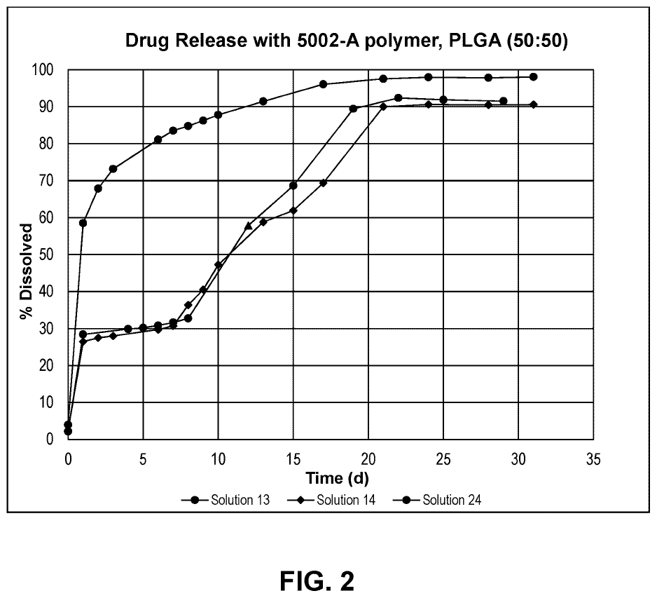

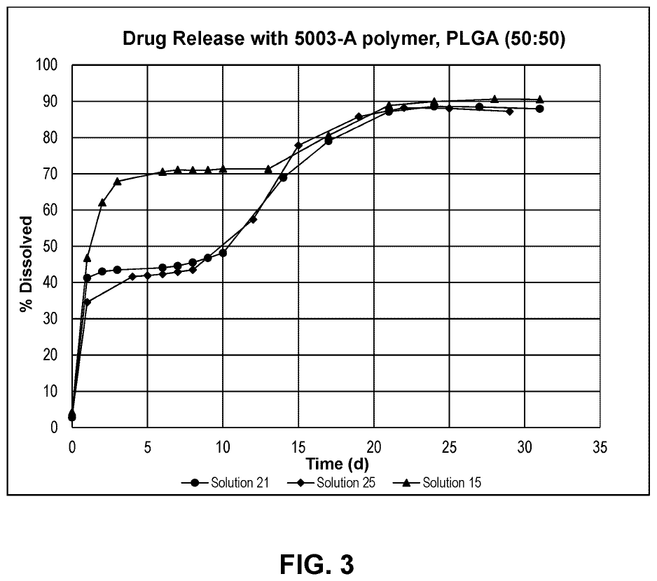

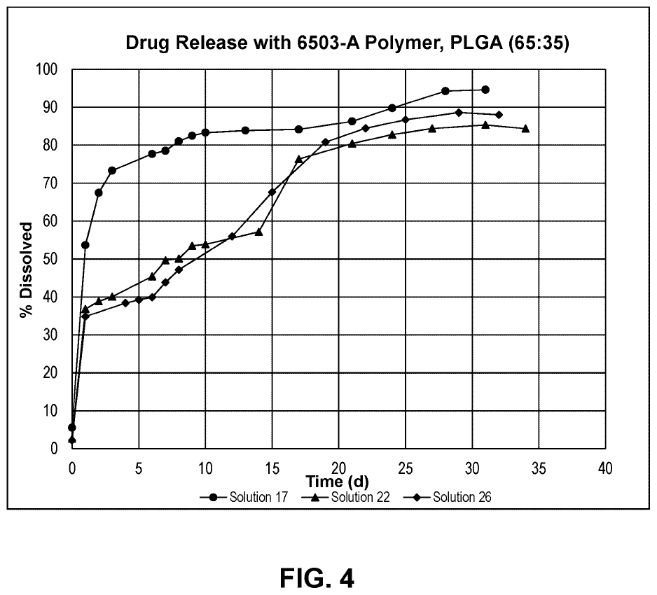

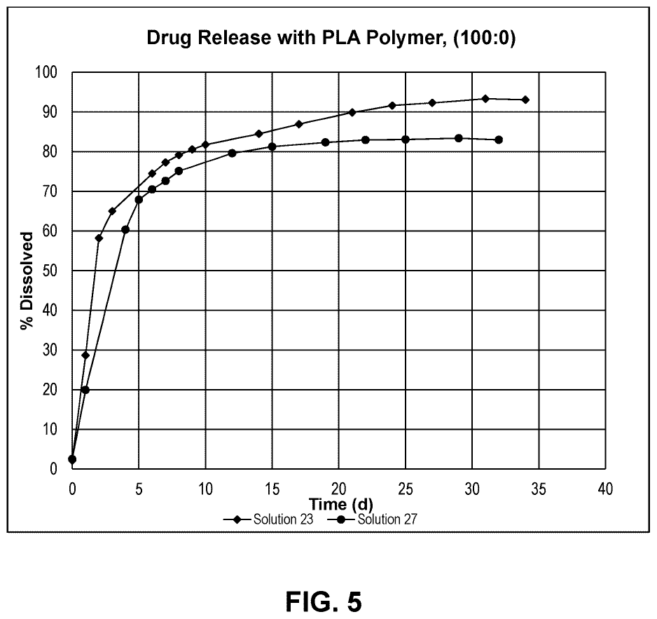

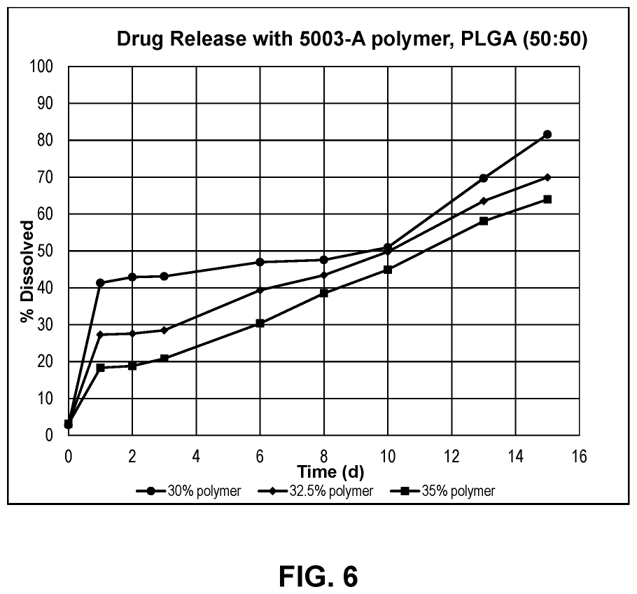

The amount of cyanocobalamin released by the solutions in Table 5 was measured by adding approximately 250 mg of the formulation solution in a glass media bottle containing pH 7.4 PBS buffer. The glass media bottles were placed in a shaking water bath that was set at 75 RPM with the temperature at 37° C. FIGS. 1-6 show the amount of cyanocobalamin released over time for solutions listed in Table 5. As shown in the FIGS. 1-6, an increase in polymer concentration from 25% to 30% decreased the initial burst release of cyanocobalamin. The figures also showed that the burst release can be decreased by lowering the ratio of lactic acid in the polymer.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1-50. (canceled)

51. A pharmaceutical long acting depot injectable composition comprising a vitamer of vitamin B12 or a pharmaceutically acceptable derivative thereof, wherein the composition is dispersed in a biodegradable carrier.

52. The composition of claim 51, wherein the vitamer is cyanocobalamin, and optionally cyanocobalamin pamoate.

53. The composition of claim 51, wherein the biodegradable carrier is a polymer, optionally selected from the group consisting of polylactides, polyglycolides, poly(lactide-co-glycolide) s, polycaprolactones, polydioxanones, polycarbonates, polyhydroxybutyrates, poly(3-hydroxybutyrate-co-3-hydroxyvalerate), tri (ethylene glycol) poly(orthoester), polyalkylene oxalates, polyanhydrides, polyamides, polyesteramides, polyurethanes, polyacetals, polyorthocarbonates, polyphosphazenes, polyhydroxyvalerates, polyalkylene succinates, polyorthoesters, and copolymers, block copolymers, branched copolymers, terpolymers, and combinations and mixtures thereof.

54. The composition of claim 53, wherein the polymer is a polylactide, a poly(lactide-co-glycolide), or a combination thereof, optionally wherein the poly(lactide-co-glycolide) has a lactide to glycolide ratio of from about 5:95 to about 95:5.

55. The composition of claim 53, wherein the polymer has an intrinsic viscosity of from about 0.1 dL/g to about 1 dL/g, and/or wherein the polymer has a molecular weight of from about 15 kDa to about 35 kDa.

56. The composition of claim 53, wherein the concentration of the polymer in the composition is from about 10% to about 40%.

57. The composition of claim 51, wherein the composition is stable for at least about 6 months at 25° C.

58. The composition claim 51, wherein the amount of the vitamer of vitamin B12 in the composition is from about 0.5 mg to about 3 mg.

59. The composition of claim 51, wherein the concentration of the vitamer of vitamin B12 in the composition is from about 1 mg/g to about 6 mg/g.

60. The composition of claim 51, wherein the composition comprises one or more pharmaceutically acceptable excipients selected from the group consisting of polymers, salts, solvents, vehicles and combinations thereof.

61. The composition of claim 60, wherein the solvent is a non-aqueous solvent.

62. The composition of claim 61, wherein the non-aqueous solvent is a water-miscible solvent.

63. The composition of claim 62, wherein the water-miscible solvent is selected from the group consisting of dimethyl sulfoxide (DMSO), N-methyl-2-pyrrolidone (NMP), triacetin, propylene glycol, and combinations thereof.

64. The composition of claim 61, wherein the non-aqueous solvent solvent is a water-immiscible solvent.

65. The composition of claim 64, wherein the water-immiscible solvent is selected from the group consisting of benzyl alcohol, benzyl benzoate, and combinations thereof.

66. The composition of claim 51, wherein the composition is formulated for parenteral administration.

67. A method for treating vitamin B12 deficiency in a subject, comprising administering to the subject a therapeutically effective amount of the composition of claim 1, wherein the subject's vitamin B12 levels remain at therapeutically effective levels over a period of about two to about six months.

68. The method of claim 67, wherein the administration is intramuscular or subcutaneous.

69. The method of claim 67, wherein the composition is administered once every 0.5 to 6 months.

70. The method of claim 67, wherein the composition provides a therapeutic dose of cyanocobalamin for at least 12 weeks.
